(19) United States
(12) Patent Application Publication
(10) Pub. No.: US 2011/0280851 A1
(43) Pub. Date: Nov. 17, 2011

(54) Compositions Comprising Cranberry Extract and Methods of Use Thereof

(76) Inventors: Alexander S. Herzlinger, Exton, PA (US); Tina Shih-Ping Wu, Glendale, AZ (US)

(21) Appl. No.: 13/106,202

(22) Filed: May 12, 2011

Related U.S. Application Data
(60) Provisional application No. 61/333,922, filed on May 12, 2010.

(51) Int. Cl. A61K 36/45; A61K 33/06; A61K 33/26; A61K 33/04; A61P 13/02; A61K 36/71; A61K 36/8945; A61K 36/06; A61P 31/00; A61P 31/04; A61K 38/43; A61K 36/82

(52) U.S. Cl. 424/94.1; 424/732; 424/682; 424/646; 424/702; 424/729; 424/195.16

(57) ABSTRACT

Described herein are compositions that comprise cranberry extract and are potent and low in calories. Methods of preventing or treating infections by administering the described compositions are also provided.

### RELATED APPLICATIONS

This application claims the benefit of priority to United States Provisional Patent Application Ser. No. 61/333,922, filed May 12, 2010, the contents of which are hereby incorporated by reference.

### BACKGROUND OF THE INVENTION

Urinary tract infections (UTIs) account for over 11.3 million office visits and 1.5 million emergency department visits per year. These statistics become even more staggering when combined with the fact that fewer than 40% of women seek clinical care for what they believe are UTIs. Approximately 13.3% of women suffer from UTIs each year. Over 50% of women develop at least one UTI in their lives.

Common symptoms of a UTI include urinary frequency and pain. While uncomplicated UTIs in women are usually benign in nature, a UTI elevates the risk of premature delivery and fetal mortality among pregnant women. Furthermore, a simple UTI can progress to pyelonephritis (kidney infection), bacteremia (bacteria in the blood), sepsis (whole body infection), or death. In elderly patients, UTIs are a significant cause of mortality; up to 3% of elderly patients who develop pyelonephritis die from their UTI.

The urinary tract is comprised of the kidneys, ureters, bladder, and urethra. The kidneys are a pair of organs that help to eliminate waste and excess fluid from the blood in the form of urine. The ureters drain the urine from the kidneys to the bladder. The bladder stores the urine until one is ready to void. When voiding, the urine is released from the bladder out of the body through a tube called the urethra. Women have a short urethra, while men have a longer urethra, making women significantly more susceptible to UTIs.

There are four general epicenters that make women more susceptible to UTIs:
Sexual Activity: Young (age 18-24), sexually active women are three times more likely to develop UTIs than non sexually active women. Sexual activity can introduce E. coli into women's urinary tracts and many young women have not developed proper post-sexual activity hygiene.
Pregnancy: Pregnant women become more at-risk for UTIs due to the pressure that the uterus asserts on the bladder and pregnant women's compromised immune systems.
Menopause: Women experience anatomical changes during menopause that thin out vaginal membranes, weakening post-menopausal women's anti-bacterial defense mechanisms.
Incontinence: At least ⅓ of women over 65 experience urge or stress incontinence. Incontinence increases the reproduction of bacteria in the vaginal area, increasing the likelihood of UTIs.

The cause of urinary tract infections is well-documented with over 85% of infections due to Escherichia coli (E. coli) bacteria. E. coli normally live in the colon and do not cause infections when they remain in the colon. The urinary tract and urine is normally sterile. There are a number of ways that E. coli can find their way to the urinary tract, most commonly through sexual intercourse, poor hygiene, or catheters.

E. coli are oblong bacteria with finger-like appendages called fimbriae that allow them to be mobile. A small amount of E .coli can invade the urethra, and then crawl into the bladder. The bacteria then multiply very rapidly and begin to release toxins that cause the bladder to spasm. When the bladder begins to spasm, the patient feels pain, urgency, and urinates multiple times an hour. A colony count of greater than 100,000 colony forming units (CFU)/mL generally indicates a UTI. The E. coli can multiply to 100,000 CFU/mL overnight. A “simple” UTI does not progress past the bladder; a “complicated” UTI occurs when the simple UTI ascends past the bladder. If untreated, the bacteria then can continue to ascend to the kidneys causing pyelonephritis (kidney infection).

UTIs are an enormous cost to the healthcare system. Community-acquired UTIs continue to be a leading cause of infection in all ages. With approximately 11.3 million office visits in the US per year, the costs are staggering. UTIs comprise 40% of hospital-acquired infections, making it the most common hospital acquired infection.

Clinicians and women have long associated cranberries with UTI prevention. Until the late 1990s, clinicians assumed that the cranberries' acidic properties created a harmful environment for E. coli. Research conducted over the past decade has elucidated cranberries' mechanism of action for UTI prevention.

Cranberries contain A-type proanthocyanidins (PACs)—the active ingredient for UTI prevention. Proanthocyanidins prevent the adherence of E. coli fimbriae to the urinary tract wall, thereby blocking E. coli from sticking to the bladder wall. In addition to their antibacterial properties, PACs may also possess anticancer activities or significant cardiac benefits, including decreased levels of atherosclerosis.

Studies suggest that 30 mg PACs every 12 hours represents the optimal dose for UTI prevention. However, due to many factors, including calories, sugar load, and others, side effects are common when existing cranberry products are used at the recommended dosages. Cranberry juice cocktail (typically 27% juice) represents one of the most caloric drinks available, with 137 calories per 8 fl. Oz.—that's 37% more calories than regular cola. The high levels of sugar and calories in cranberry juice cocktails prove especially problematic for UTI sufferers who also suffer from diabetes or gestational diabetes. Similarly, the low pH of cranberry juice cocktails could be problematic for patients also suffering from gastrointestinal problems, such as ulcers.

Diet cranberry juice products are heavily diluted (5-7% juice) and laden with artificial ingredients and sweeteners. Diet cranberry products contain far fewer antibiotics than clinical studies suggest. Similarly, cranberry pills have failed to capture consumer loyalty because they contain scant active ingredients. Cranberry pills also suffer from low consumer awareness. Furthermore, cranberry pills fail to promote hydration and flushing; hydration is critical during an acute infection and for prevention.

There exists a need for a potent, effective, low-calorie, natural product that is optimized for women's health.

### SUMMARY OF THE INVENTION

In certain embodiments, the invention relates to a composition comprising

dried cranberry extract;

a pharmaceutically acceptable carrier;

a flow agent;

a first vitamin;

a flavor ingredient;

a preservative; and

an emulsion stabilizer.

In certain embodiments, the invention relates to a composition comprising

dried cranberry extract;

a pharmaceutically acceptable carrier, wherein the pharmaceutically acceptable carrier comprises magnesium hydroxide;

a flow agent, wherein the flow agent comprises tricalcium phosphate;

a first vitamin, wherein the first vitamin comprises ascorbic acid;

a flavor ingredient;

a preservative, wherein the preservative comprises citric acid; and

an emulsion stabilizer, wherein the emulsion stabilizer comprises starch.

In certain embodiments, the invention relates to a method of treating or preventing an infection in a mammal, comprising administering to a subject in need thereof a therapeutically effective amount of any one of the aforementioned compositions.

### BRIEF DESCRIPTION OF THE FIGURES

FIG. 1 tabulates the components of an exemplary composition of the invention.

FIG. 2 depicts a comparison between the number of cranberry equivalents per serving of a composition of the invention (left bar) with the number of cranberry equivalents per dose of various commercial cranberry supplements.

FIG. 3 depicts a comparison of the number of calories per dose of a composition of the invention (left bar) with the number of calories per dose of various commercial cranberry juice cocktails.

FIG. 4 tabulates the advantages of compositions of the invention (right column) over 100% cranberry juice and various cranberry juice cocktails.

FIG. 5 depicts a comparison between observed urinary anti-adhesion activity recorded per time period by 10 study participants (dried cranberry extract (CystiCran) (top graph, left bars) and 27% cranberry juice cocktail (CJC) (top graph, right bars)).

FIG. 6 depicts a comparison of observed urinary anti-adhesion activity for each participant over time (dried cranberry extract (CystiCran) and 27% cranberry juice cocktail (CJC)).

FIG. 7 depicts the total observed urinary anti-adhesion activity recorded for men and for women for dried cranberry extract (CystiCran) (left bars) and 27% cranberry juice cocktail (CJC) (right bars).

FIG. 8 depicts a comparison of observed urinary anti-adhesion activity for each participant over time for (a) a composition of the invention (You-T); and (b) super strength cranberry.

FIG. 9 depicts a comparison between observed urinary anti-adhesion activity recorded per time period by 10 study participants (a composition of the invention (You-T) and super strength cranberry (SSC)).

FIG. 10 depicts a tabulated comparison of the bacterial anti-adhesion activity and PAC levels of a composition of the invention (You-T), super strength cranberry (SSC), and Cystex, as described in Example 6; the * indicates the concentration at which anti-adhesion activity could be detected.

### DESCRIPTION OF THE INVENTION

### Overview

In certain embodiments, the invention relates to a composition comprising cranberry extract that is useful in the prevention and treatment of urinary tract infections, or in the promotion of urinary tract health. In certain embodiments, the compositions are low in calories, taste great, or are all-natural. In certain embodiments, the compositions comprise about 30 mg of PACs per serving. In certain embodiments, this is the equivalent of greater than 3 lbs. of cranberries. In certain embodiments, the compositions are over 400 times more powerful in terms of cranberry equivalence than the leading cranberry supplement.

### Definitions

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “extract” refers to a product prepared by extraction. The extract may be in the form of a solution in a solvent, or the extract may be a concentrate or essence which is free of, or substantially free of solvent. The term extract may be a single extract obtained from a particular extraction step or series of extraction steps or the extract also may be a combination of extracts obtained from separate extraction steps. For example, extract “a” may be obtained by extracting cranberry with alcohol in water, while extract “b” may be obtained by super critical carbon dioxide extraction of cranberry. Extracts a and b may then be combined to form extract “c”. Such combined extracts are thus also encompassed by the term “extract”.

As used herein, the term “fraction” means the extract comprising a specific group of chemical compounds characterized by certain physical, chemical properties or physical or chemical properties.

The term “synergistic” is art recognized and refers to two or more components working together so that the total effect is greater than the sum of the components.

The term “treating” is art-recognized and refers to curing as well as ameliorating at least one symptom of any condition or disorder.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

As used herein, the term “microbe” refers to a microscopic organism, usually invisible to the naked eye (e.g., bacteria, yeasts).

As used herein, the term “bacterium” refers to a prokaryotic class of unicellular (single or chains) organisms or microbes that lack an defined and organized nucleus and fall into two general classes Gram-positive and Gram negative based on the chemically staining properties of their cell wall.

As used herein, the term “urinary tract infection” or “UTI” refers to a bacterial infection that affects any part of the urinary tract. When bacteria get into the bladder or kidney and multiply in the urine, they cause a UTI. The most common type of UTI is a bladder infection which is also often called cystitis.

As used herein, the term “adhesion” refers to the binding of a cell to a surface, extracellular matrix or another cell or a manmade material using cell adhesion molecules such as selectins, integrins, and cadherins or, more generally, adhesins.

As used herein, the term “biostatic” refers to molecules that inhibit growth and reproduction of bacteria without killing them.

### Exemplary Compositions of the Invention

In certain embodiments, the invention relates to a composition comprising

dried cranberry extract;

a pharmaceutically acceptable carrier;

a flow agent;

a first vitamin;

a flavor ingredient;

a preservative; and

an emulsion stabilizer.

In certain embodiments, the invention relates to a composition consisting essentially of

dried cranberry extract;

a pharmaceutically acceptable carrier;

a flow agent;

a first vitamin;

a flavor ingredient;

a preservative; and

an emulsion stabilizer.

In certain embodiments, the invention relates to a composition consisting of

dried cranberry extract;

a pharmaceutically acceptable carrier;

a flow agent;

a first vitamin;

a flavor ingredient;

a preservative; and

an emulsion stabilizer.

In certain embodiments, the invention relates to a composition comprising

dried cranberry extract;

a pharmaceutically acceptable carrier, wherein the pharmaceutically acceptable carrier comprises magnesium hydroxide;

a flow agent, wherein the flow agent comprises tricalcium phosphate;

a first vitamin, wherein the first vitamin comprises ascorbic acid;

a flavor ingredient;

a preservative, wherein the preservative comprises citric acid; and

an emulsion stabilizer, wherein the emulsion stabilizer comprises starch.

In certain embodiments, the invention relates to a composition consisting essentially of

dried cranberry extract;

a pharmaceutically acceptable carrier, wherein the pharmaceutically acceptable carrier consists essentially of magnesium hydroxide;

a flow agent, wherein the flow agent consists essentially of tricalcium phosphate;

a first vitamin, wherein the first vitamin consists essentially of ascorbic acid;

a flavor ingredient;

a preservative, wherein the preservative consists essentially of citric acid; and

an emulsion stabilizer, wherein the emulsion stabilizer consists essentially of starch.

In certain embodiments, the invention relates to a composition comprising

dried cranberry extract;

a pharmaceutically acceptable carrier, wherein the pharmaceutically acceptable carrier consists of magnesium hydroxide;

a flow agent, wherein the flow agent consists of tricalcium phosphate;

a first vitamin, wherein the first vitamin consists of ascorbic acid;

a flavor ingredient;

a preservative, wherein the preservative consists of citric acid; and

an emulsion stabilizer, wherein the emulsion stabilizer consists of starch.

In certain embodiments, the invention relates to any one of the aforementioned compositions, further comprising a second vitamin, a mineral, an antioxidant, an omega-3 fatty acid, a sugar or sugar substitute, an amino acid, an herbal extract, an enzyme, a neuronutrient, or a dietary supplement.

In certain embodiments, the invention relates to any one of the aforementioned compositions, further comprising a second vitamin, wherein the second vitamin is vitamin A, vitamin E, vitamin B6, vitamin B12, riboflavin, thiamin, niacin, vitamin K, vitamin D, or folic acid.

In certain embodiments, the invention relates to any one of the aforementioned compositions, further comprising a mineral, wherein the mineral is calcium, iron, or magnesium.

In certain embodiments, the invention relates to any one of the aforementioned compositions, further comprising an antioxidant, wherein the antioxidant is epigallocatechin gallate, xeaxanthin, bilberry, selenium, or green tea.

In certain embodiments, the invention relates to any one of the aforementioned compositions, further comprising an herbal extract, wherein the herbal extract is black cohosh or wild yam extract.

In certain embodiments, the invention relates to any one of the aforementioned compositions, further comprising a dietary supplement, wherein the dietary supplement is red yeast rice.

In certain embodiments, the invention relates to any one of the aforementioned compositions, further comprising a sugar substitute, wherein the sugar substitute is a steviol glycoside or a sugar alcohol

In certain embodiments, the invention relates to any one of the aforementioned compositions, further comprising a sugar substitute, wherein the sugar substitute is rebaudioside A or erythritol.

In certain embodiments, the invention relates to any one of the aforementioned compositions, wherein the dried cranberry extract, pharmaceutically acceptable carrier, and flow agent, taken together, are present in an amount from about 2% to about 8% by weight of the composition.

In certain embodiments, the invention relates to any one of the aforementioned compositions, wherein the dried cranberry extract, pharmaceutically acceptable carrier, and flow agent, taken together, are present in about 2%, about 3%, about 4%, about 5%, about 6%, about 7%, or about 8% by weight of the composition.

In certain embodiments, the invention relates to any one of the aforementioned compositions, wherein the dried cranberry extract comprises proanthocyanidins in an amount from about 20% to about 40% by weight of the dried cranberry extract.

In certain embodiments, the invention relates to any one of the aforementioned compositions, wherein the dried cranberry extract comprises proanthocyanidins in about 20%, about 22%, about 24%, about 26%, about 28%, about 30%, about 32%, about 34%, about 36%, about 38%, or about 40% by weight of the dried cranberry extract.

In certain embodiments, the invention relates to any one of the aforementioned compositions, wherein the dried cranberry extract comprises phenolic compounds in an amount from about 30% to about 50% by weight of the dried cranberry extract.

In certain embodiments, the invention relates to any one of the aforementioned compositions, wherein the dried cranberry extract comprises phenolic compounds in about 30%, about 32%, about 34%, about 36%, about 38%, about 40%, about 42%, about 44%, about 46%, about 48%, or about 50% by weight of the dried cranberry extract.

In certain embodiments, the invention relates to any one of the aforementioned compositions, wherein the dried cranberry extract comprises water in an amount from about 1.5% to about 4.5% by weight of the dried cranberry extract.

In certain embodiments, the invention relates to any one of the aforementioned compositions, wherein the dried cranberry extract comprises water in about 1.5%, about 2.0%, about 2.5%, about 3.0%, about 3.5%, about 4.0%, or about 4.5% by weight of the dried cranberry extract.

In certain embodiments, the invention relates to any one of the aforementioned compositions, wherein the bulk density of the dried cranberry extract is from about 0.25 g/mL to about 0.75 g/mL, as measured by USP <616>, method I.

In certain embodiments, the invention relates to any one of the aforementioned compositions, wherein the bulk density of the dried cranberry extract is about 0.25 g/mL, about 0.30 g/mL, about 0.35 g/mL, about 0.40 g/mL, about 0.45 g/mL, about 0.50 g/mL, about 0.55 g/mL, about 0.60 g/mL, about 0.65 g/mL, about 0.70 g/mL, or about 0.75 g/mL.

In certain embodiments, the invention relates to any one of the aforementioned compositions, wherein the pH of the dried cranberry extract in a 10% w/v solution with ethanol is from about 3.0 to about 5.0.

In certain embodiments, the invention relates to any one of the aforementioned compositions, wherein the pH of the dried cranberry extract in a 10% w/v solution with ethanol is about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0.

In certain embodiments, the invention relates to any one of the aforementioned compositions, wherein the first vitamin is present in an amount from about 0.4% to about 1.2% by weight of the composition.

In certain embodiments, the invention relates to any one of the aforementioned compositions, wherein the first vitamin is present in about 0.4%, about 0.6%, about 0.8%, about 1.0%, or about 1.2% by weight of the composition.

In certain embodiments, the invention relates to any one of the aforementioned compositions, wherein the flavor ingredient is present in an amount from about 70% to about 95% by weight of the composition.

In certain embodiments, the invention relates to any one of the aforementioned compositions, wherein the flavor ingredient is present in about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95% by weight of the composition.

In certain embodiments, the invention relates to any one of the aforementioned compositions, wherein the flavor ingredient is mixed berry, blueberry, cranberry-pomegranate, or cranberry-acai.

In certain embodiments, the invention relates to any one of the aforementioned compositions, wherein the preservative is present in an amount from about 1.5% to about 4.5% by weight of the composition.

In certain embodiments, the invention relates to any one of the aforementioned compositions, wherein the preservative is present in about 1.5%, about 2.0%, about 2.5%, about 3.0%, about 3.5%, about 4.0%, or about 4.5% by weight of the composition.

In certain embodiments, the invention relates to any one of the aforementioned compositions, wherein the emulsion stabilizer is present in an amount from about 3.5% to about 11% by weight of the composition.

In certain embodiments, the invention relates to any one of the aforementioned compositions, wherein the emulsion stabilizer is present in about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight of the composition.

In certain embodiments, the invention relates to any one of the aforementioned compositions, wherein the composition is in the form of a powder.

In certain embodiments, the invention relates to any one of the aforementioned compositions, wherein the composition is in the form of a tablet.

In certain embodiments, the invention relates to any one of the aforementioned compositions, wherein the composition is in the form of a liquid or gel.

In certain embodiments, the invention relates to any one of the aforementioned compositions, wherein a serving of the composition is about 5 g, about 6, g, about 7 g, about 8 g, about 9 g, or about 10 g.

In certain embodiments, the invention relates to any one of the aforementioned compositions, wherein the composition has from about 5 to about 30 calories per serving.

In certain embodiments, the invention relates to any one of the aforementioned compositions, wherein the composition has about 5, about 10, about 15, about 20, about 25, or about 30 calories per serving.

In certain embodiments, the invention relates to any one of the aforementioned compositions, wherein the dried cranberry extract comprises from about 15 mg to about 40 mg proanthocyanidins per serving.

In certain embodiments, the invention relates to any one of the aforementioned compositions, wherein the dried cranberry extract comprises about 15 mg, about 20 mg, about 25 mg, about 30 mg, about 35 mg, or about 40 mg proanthocyanidins per serving.

In certain embodiments, the invention relates to any one of the aforementioned compositions, wherein the dried cranberry extract comprises from about 40 mg to about 100 mg proanthocyanidins per serving.

In certain embodiments, the invention relates to any one of the aforementioned compositions, wherein the dried cranberry extract comprises about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg proanthocyanidins per serving.

In certain embodiments, the invention relates to any one of the aforementioned compositions, wherein the composition is a food composition

In certain embodiments, the invention relates to any one of the aforementioned compositions, wherein the composition is a pharmaceutical composition

In certain embodiments, the invention relates to any one of the aforementioned compositions, wherein the composition is formulated as a functional food, dietary supplement, powder, or beverage.

In certain embodiments, the invention relates to any one of the aforementioned compositions, in a packet.

In certain embodiments, the invention relates to any one of the aforementioned compositions, wherein the packet is a stick pack or a sachet.

In certain embodiments, the invention relates to any one of the aforementioned compositions, wherein the composition is in admixture an amount of water.

In certain embodiments, the compositions of the invention may be formulated in forms such as a paste, powder, oils, liquids, suspensions, solutions, ointments, effervescent tablets, chews, or other forms, comprising, one or more fractions or sub-fractions to be used as dietary supplements, nutraceuticals, or such other preparations that may be used to prevent or treat various human ailments. The extracts can be processed to produce such consumable items, for example, by mixing them into a food product, in a capsule or tablet, or providing the paste itself for use as a dietary supplement, with sweeteners or flavors added as appropriate. Accordingly, such preparations may include, but are not limited to, cranberry extract preparations for oral delivery in the form of tablets, capsules, lozenges, liquids, emulsions, dry flowable powders and rapid dissolve tablets. The cranberry extracts may advantageously be formulated into a suppository or lozenge for vaginal administration.

In one embodiment, a dry extracted cranberry composition is mixed with a suitable solvent, such as but not limited to water or ethyl alcohol, along with a suitable food-grade material using a high shear mixer and then spray air-dried using conventional techniques to produce a powder having grains of cranberry extract particles combined with a food-grade carrier.

In a particular example, cranberry extract composition is mixed with a food-grade or pharmaceutically acceptable carrier and an ethyl alcohol solvent using a high shear mixer. Inert carriers, such as silica or tricalcium phosphate, can be added to improve the flow of the final powder that is formed. The amount of ethyl alcohol used is preferably the minimum needed to form a solution with a viscosity appropriate for spray air-drying. Typical amounts are in the range of between about 5 to about 10 liters per kilogram of extracted material. The solution is spray air-dried to generate a powder with an average particle size comparable to that of the starting carrier material.

Once a dry extract powder is obtained, such as by the methods discussed herein, it can be distributed for use, e.g., as a dietary supplement or for other uses. In a particular embodiment, the novel extract powder is mixed with other ingredients to form a tableting composition of powder that can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° to about 85° C. The dried tablet can then be packaged for distribution.

Compositions can be in the form of a paste, resin, oil, powder or liquid. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

Dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

The described extracts may be combined with extracts from other plants such as, but not limited to, varieties of Gymnema, turmeric, Boswellia, guarana, cherry, lettuce, Echinacea, piper betel leaf, Areca catechu, Muira puama, ginger, willow, suma, kava, horny goat weed, Ginkgo biloba, mate, garlic, puncture vine, arctic root, astragalus, Eucommia, Cinnamomum, Cassia, and Uncaria, or pharmaceutical or nutraceutical agents.

### Exemplary Formulations of the Invention

In certain embodiments, the invention relates to a formulation comprising a serving of any one of the aforementioned compositions; and an amount of liquid.

In certain embodiments, the invention relates to any one of the aforementioned formulations, wherein the liquid is water.

### Exemplary Kits of the Invention

In certain embodiments, the invention relates to a kit, wherein the kit comprises any one of the aforementioned compositions in a packet; and instructions for use.

In certain embodiments, the invention relates to any one of the aforementioned kits, wherein the packet is a stick pack or a sachet.

### Exemplary Compositions of the Invention for Particular Uses

In certain embodiments, the invention relates to any one of the aforementioned compositions for use in the treatment or prevention of an infection.

In certain embodiments, the invention relates to any one of the aforementioned compositions for use in the promotion of urinary tract health.

### Exemplary Methods of the Invention

In certain embodiments, the invention relates to a method of treating an infection in a mammal, comprising administering to a subject in need thereof a therapeutically effective amount of any one of the aforementioned compositions.

In certain embodiments, the invention relates to any one of the aforementioned methods, wherein the infection is a bacterial infection.

In certain embodiments, the invention relates to any one of the aforementioned methods, wherein the infection is E. coli

In certain embodiments, the invention relates to any one of the aforementioned methods, wherein the composition is administered for a time sufficient to reduce or eliminate symptoms associated with the infection.

In certain embodiments, the invention relates to any one of the aforementioned methods, wherein the composition is administered twice a day.

In certain embodiments, the invention relates to any one of the aforementioned methods, wherein the composition is administered twice a day for one week.

In certain embodiments, the invention relates to a method of preventing an infection in a mammal, comprising administering to a subject in need thereof a therapeutically effective amount of any one of the aforementioned compositions.

In certain embodiments, the invention relates to any one of the aforementioned methods, wherein the infection is a bacterial infection.

In certain embodiments, the invention relates to any one of the aforementioned methods, wherein the infection is E. coli.

In certain embodiments, the invention relates to any one of the aforementioned methods, wherein the composition is administered for a time sufficient to prevent symptoms associated with the infection.

In certain embodiments, the invention relates to a method of promoting urinary tract health, comprising administering to a subject in need thereof a therapeutically effective amount of any one of the aforementioned compositions.

In certain embodiments, the invention relates to any one of the aforementioned methods, wherein the composition is administered once a day.

In certain embodiments, the invention relates to any one of the aforementioned methods, wherein the composition is administered one dose every other day.

In certain embodiments, the invention relates to any one of the aforementioned methods, further comprising the step of mixing the composition with an amount of water.

In certain embodiments, the invention relates to any one of the aforementioned methods, wherein the amount of water is from about 6 fl. oz. to about 12 fl. oz.

In certain embodiments, the invention relates to any one of the aforementioned methods, wherein the amount of water is about 7 fl. oz., about 8 fl. oz., about 9 fl. oz., about 10 fl. oz., or about 11 fl. oz.

In certain embodiments, the invention relates to a method of treating or preventing atherosclerosis in a mammal, comprising administering to a subject in need thereof a therapeutically effective amount of any one of the aforementioned compositions.

In certain embodiments, the invention relates to a method of lowering LDL cholesterol in a mammal, comprising administering to a subject in need thereof a therapeutically effective amount of any one of the aforementioned compositions.

In certain embodiments, the invention relates to a method of reducing or preventing oxidation of cells in a mammal, comprising administering to a subject in need thereof a therapeutically effective amount of any one of the aforementioned compositions.

In certain embodiments, the invention relates to a method of treating or preventing cancer in a mammal, comprising administering to a subject in need thereof a therapeutically effective amount of any one of the aforementioned compositions.

In certain embodiments, the invention relates to a method of treating or preventing osteoporosis in a mammal, comprising administering to a subject in need thereof a therapeutically effective amount of any one of the aforementioned compositions.

In certain embodiments, the invention relates to any one of the aforementioned methods, wherein said mammal is a human.

### EXEMPLIFICATION

### Example 1

### Composition of the Invention

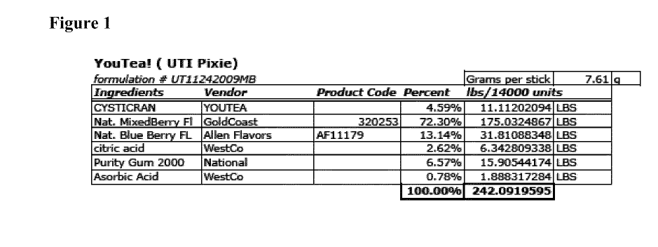

FIG. 1 depicts an exemplary formulation of the invention.

### Example 2

### Potency of Compositions of the Invention in Comparison to Supplements

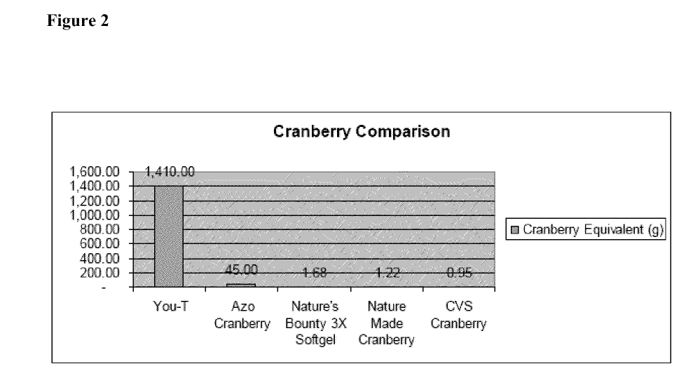

The number of cranberry equivalents were compared (FIG. 2).

### Example 3

### Downfalls of 100% Cranberry Juice

100% cranberry juice (unsweetened, found only at natural grocers) delivers a potent dose of PACs. However, taste represents this product's biggest weakness. As one cranberry researcher stated, “that stuff will take the enamel off your teeth.” Other comments from taste-testers included:

“nasty, too nasty to drink”

“Sour, gag reflex”

“Like sucking on a cranberry lemon”

“Tastes like licking a lemon—so sour”

“Too sour”

“Cranberry compound+water=gross”

“gagging”

### Example 4

### Potency of Compositions of the Invention in Comparison to Cranberry Juice Cocktail

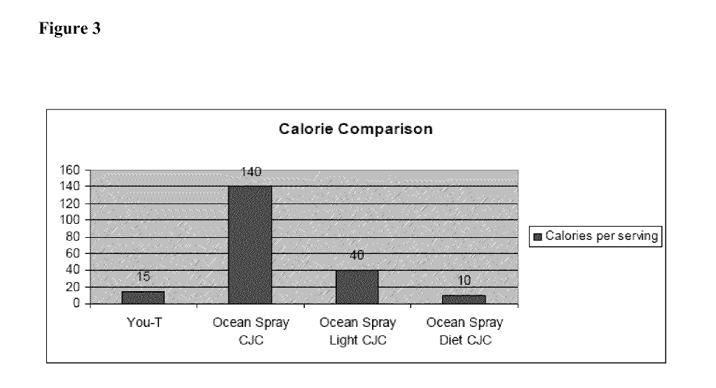

Cranberry juice cocktail typically contains 27% cranberry juice. Cranberry juice cocktail does deliver a potent dose of PACs, with about 29 mg PACs per serving. However, cranberry juice cocktail is laden with calories and sugar. An 8 oz. serving of cranberry juice cocktail contains 137 calories and 30 g (over 7 tsp) sugar. Juice manufacturers attempt to overcome cranberry juice cocktail's high calorie content with artificial sweeteners and water. For instance, Ocean Spray launched Diet Cranberry Juice Cocktail with 10 calories per serving and Light Cranberry Juice Cocktail with 40 calories per serving. However, these products are diluted and lose Cranberry Juice Cocktail's effectiveness. A comparison of the calorie content is shown in FIG. 3.

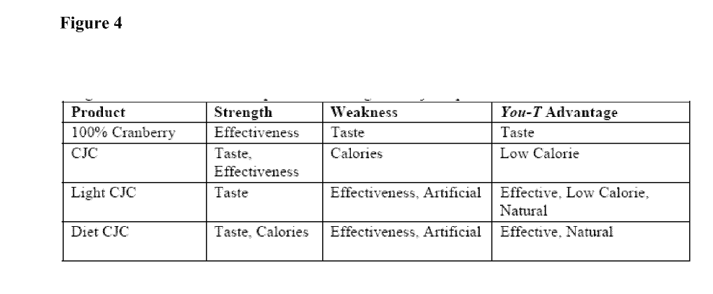

FIG. 4 summarizes the advantages of compositions of the present invention (right column) over existing cranberry products.

### Example 5

### Bacterial Anti-adhesion Activity of Human Urine: Dried Cranberry Extract (360 mg) vs. 27% Cranberry Juice Cocktail

### Objective:

Determine the ex vivo uropathogenic bacterial (P-type E. coli) anti-adhesion activity in human urine following consumption of two treatments in succession with a wash-out period between each treatment: 1) 360 mg CystiCran capsule (dried cranberry extract in a capsule), 2) 300 mL of 27% Cranberry Juice Cocktail (Ocean Spray), measured over a 36-h time frame with product consumed for two days at the beginning of the test period only.

Ex vivo Urine Study Method:

Pre-Visit Subject Preparation:

Participant inclusion and exclusion criteria: 5 women and 5 men, healthy, between the ages of 25 and 60, no current urinary infections, no diabetes, or antibiotic use for 6 months.

Dietary restrictions: participants refrained from consuming all cranberry, blueberry, pomegranate, grape, chocolate, and other high-flavonoid products for a 3-day wash out period prior to consuming test products and throughout testing period.

Study Design
3-day wash out period prior to consuming test products and throughout test period
On urine collection days, additional fluid consumption standardized participants to 240 mL every 3 hours to avoid dilution of urine samples and allow for detection of anti-adhesion activity, if present
On test days, products were administered in the morning
Urine (approximately 25 mL) was collected (clean-catch) by each participant prior to product consumption (time 0) and at 3, 6, 9, 24 and 36 hrs following product consumption
Urine was centrifuged, filtered (0.45 micron filter) and immediately frozen at −20° C.

Urine Protocol Specifics

Background urine samples were taken from all 10 participants prior to consumption of treatment products. Treatment 1 (one 360 mg CystiCran capsule) was administered in the morning over a 2-day period. On the morning of day 2, following CystiCran ingestion, urine was collected after product consumption and immediately frozen at −20° C. After a 3-day wash-out period, a background urine was collected and treatment 2 (300 mL of 27% Cranberry Juice Cocktail (CJC)) was administered, as stated above. Urine was collected at hour 3, 6, 9, 24 and 36 and frozen at −20° C. hemagglutination assay specific for uropathogenic P-fimbriated E. coli according to Foo et al. (Phytochemistry, 2000). A 30-μL drop of each urine was incubated with 10 μL of bacterial suspension on a 24-well polystyrene plate for 10 min at room temperature on a rotary shaker. Freshly drawn HRBCs (Al, Rh+) were suspended (3%) in PBS and added separately (10-μL drops) to test suspensions, which were then incubated for 20 min on a rotary shaker at room temperature and evaluated microscopically for the ability to prevent agglutination.

Anti-adhesion activity of each urine sample was scored visually based on a quantitative estimation of percent agglutination of each sample using the following scale:

0=no anti-adhesion activity,

1=50% anti-adhesion activity,

2=100% anti-adhesion activity.

A score of 2 indicates significant anti-adhesion activity in the urine, whereas a score of 1 indicates moderate activity. The detection limits of the anti-adhesion assay are not high enough to allow quantification of the activity in each urine sample via a dilution series; therefore the result is presented as either a positive or a negative for the activity of each sample. Anti-adhesion assays were repeated four times per sample and the results averaged. Controls included wells containing

bacteria+PBS,

HRBC+PBS,

bacteria+test material,

HRBC+test material, and

bacteria+HRBC.

Data were analyzed statistically using ANOVA.

### Results and Discussion:

No anti-adhesion activity was detected in urines prior to product consumption. Urinary pH averaged 6.5, eliminating a bacteriostatic effect.

360 mg CystiCran vs. 300 mL CJC: Summing all observed anti-adhesion activity recorded for all participants over every time period yielded 48 out of a possible 120 for CystiCran, and 41/120 for CJC.

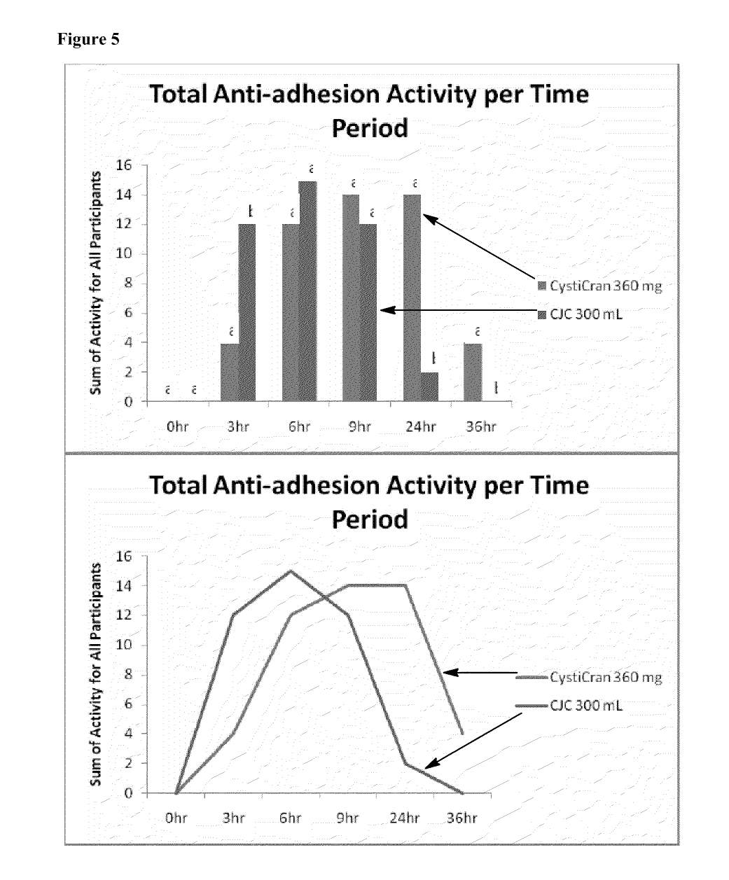

The differences between the products were not statistically significant. By time period, the post-CJC urinary activity was significantly greater (P Value 0.0015) at the 3-hr time period than the activity for CystiCran, whereas at 24 and 36 hours CystiCran was significantly greater than CJC (p values 0.002 and 0.025, respectively) (FIG. 5). This suggests that CJC has a more rapid and substantial effect in the first 3-6 hours, which it maintains at 9 hours, but diminishes thereafter. Peak activity for CJC is at 6 hours. The CystiCran activity is substantial from 6-24 hours and reaches peak activity at 9 hours. CystiCran had some residual activity at 36 hours. CystiCran is a powdered product and may take longer to metabolize than the juice, which could explain the shift in the pharmacokinetic patterns. Further research is needed to determine what activity levels at each time period correspond to a biologically relevant decrease in urinary tract infections.

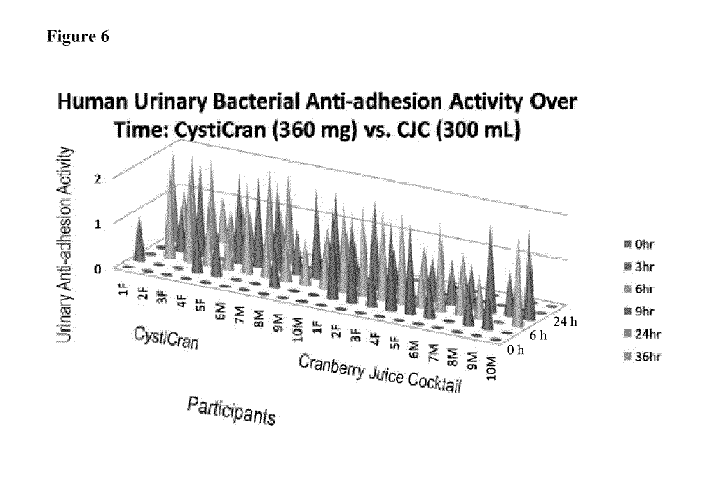

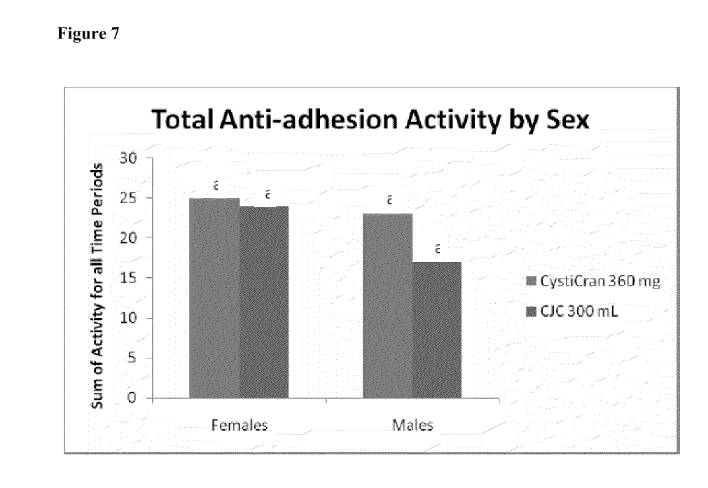

The overall data for all participants at each time period is presented in FIG. 6. Women responded similarly to each product, as did men (FIG. 7).

Overall summary

There was no significant difference in overall ex vivo urinary bioactivity between CJC and CystiCran. CJC had a significantly higher spike of activity in the first 6 hours following ingestion, whereas CystiCran spiked at 9 hours and then began to drop off, however some residual activity was evident at 36 hours post-ingestion. The activity at 36 hours was minimal and may not be sufficient to prevent bacterial adhesion. Additional work needs to be done to determine the level of activity needed at each time period to prevent UTIs.

### Example 6

### Bacterial Anti-adhesion Activity of Human Urine: Composition of the Invention vs. Super Strength Cranberry

The study summarized in Example 5 was repeated with a composition of the invention (You-T) and super strength cranberry. Super strength cranberry (SSC) may be, for example, Organic CranRich® Cranberry Concentrate 36:1 (Vaccinium macrocarpon) (powder) (fruit).500 mg; Non-medicinal ingredients: Softgel (gelatin, glycerin, purified water), organic flaxseed oil, non-GMO sunflower lecithin, organic yellow beeswax.

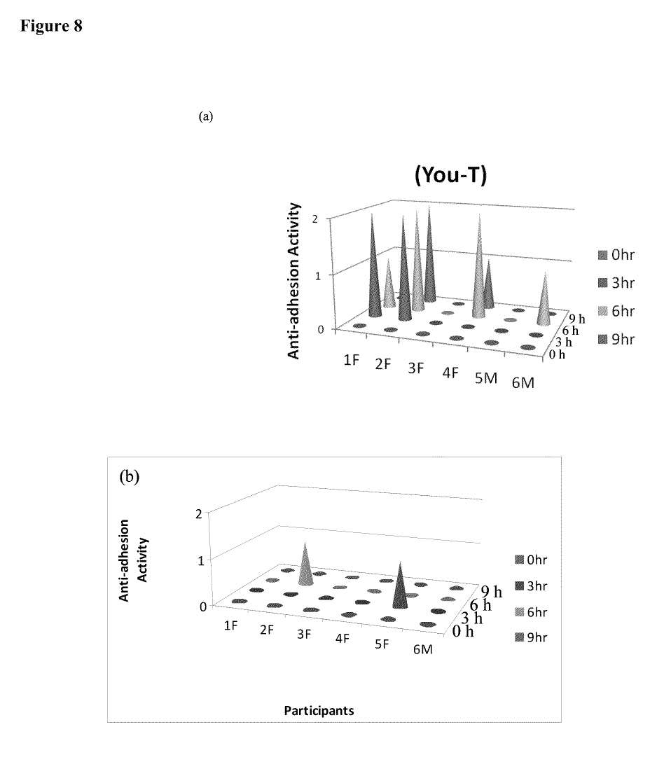

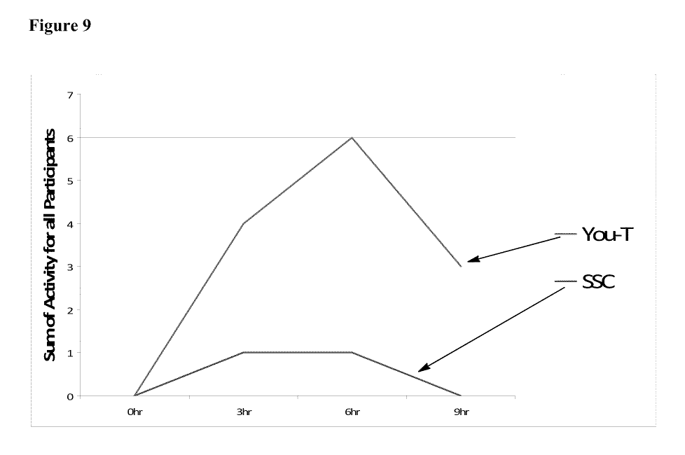

Results of the ex vivo urine study are summarized in FIG. 8 and FIG. 9.

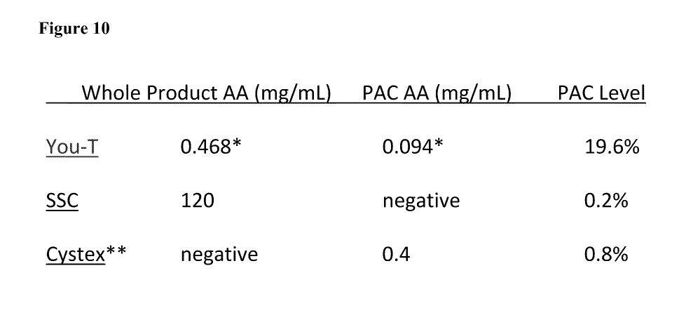

FIG. 10 depicts a tabulated comparison between the anti-adhesion properties of the two compared products, and a third product (Cystex: Cystex Liquid Cranberry Complex, marketed by DSE Healthcare, comprises cranberry concentrate, D-mannose, bromelain, and vitamin C). Cystex claims that one 15-mL serving contains 1937 mg of Proantinox. The PAC content of Proantinox is 0.4%, which is not sufficient to elicit bacterial anti-adhesion activity against P-fimbriated E. coli in vivo. Indeed, FIG. 10 shows that Cystex does not exhibit anti-adhesion.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition comprising
dried cranberry extract;
a pharmaceutically acceptable carrier, wherein the pharmaceutically acceptable carrier comprises magnesium hydroxide;
a flow agent, wherein the flow agent comprises tricalcium phosphate;
a first vitamin, wherein the first vitamin comprises ascorbic acid;
a flavor ingredient;
a preservative, wherein the preservative comprises citric acid; and
an emulsion stabilizer, wherein the emulsion stabilizer comprises starch.

2. The composition of claim 1, further comprising a second vitamin, a mineral, an antioxidant, an omega-3 fatty acid, a sugar or sugar substitute, an amino acid, an herbal extract, an enzyme, a neuronutrient, or a dietary supplement.

3. The composition of claim 1, further comprising a second vitamin, wherein the second vitamin is vitamin A, vitamin E, vitamin B6, vitamin B12, riboflavin, thiamin, niacin, vitamin K, vitamin D, or folic acid.

4. The composition of claim 1, further comprising a mineral, wherein the mineral is calcium, iron, or magnesium.

5. The composition of claim 1, further comprising an antioxidant, wherein the antioxidant is epigallocatechin gallate, xeaxanthin, bilberry, selenium, or green tea.

6. The composition of claim 1, further comprising an herbal extract, wherein the herbal extract is black cohosh or wild yam extract.

7. The composition of claim 1, further comprising a dietary supplement, wherein the dietary supplement is red yeast rice.

8. The composition of claim 1, further comprising a sugar substitute, wherein the sugar substitute is a steviol glycoside or a sugar alcohol.

9. The composition of claim 1, wherein the dried cranberry extract, pharmaceutically acceptable carrier, and flow agent, taken together, are present in an amount from about 2% to about 8% by weight of the composition.

10. The composition of claim 1, wherein the dried cranberry extract comprises proanthocyanidins in an amount from about 20% to about 40% by weight of the dried cranberry extract.

11. The composition of claim 1, wherein the dried cranberry extract comprises phenolic compounds in an amount from about 30% to about 50% by weight of the dried cranberry extract.

12. The composition of claim 1, wherein the composition is in the form of a powder, a tablet, or a liquid or gel.

13. A formulation comprising
a serving of a composition of claim 1; and
an amount of water.

14. A kit, wherein the kit comprises a composition of claim 1 in a packet; and instructions for use.

15. A method of treating or preventing an infection in a mammal, comprising administering to a subject in need thereof a therapeutically effective amount of a composition of claim 1.

16. The method of claim 15, wherein the infection is a bacterial infection.

17. The method of claim 15, wherein the infection is E. coli

18. A method of promoting urinary tract health, comprising administering to a subject in need thereof a therapeutically effective amount of a composition of claim 1.
